(19) United States
(12) Patent Application Publication
(10) Pub. No.: US 2005/0053515 A1
(43) Pub. Date: Mar. 10, 2005

(54) Cabin air quality system

(75) Inventors: Stephen F. Yates, Arlington Heights, IL (US); Felipe A. Fuentes, Harbor City, CA (US); Peter M. Michalakos, Chicago, IL (US); Chin H. Chang, Palatine, IL (US); Brian C. Krafthefer, Stillwater, MN (US); Mark Kaiser, Arlington Heights, IL (US)

Correspondence Address: Honeywell International, Inc., Law Dept. AB2, P.O. Box 2245, Morristown, NJ, 07962-9806, US

(73) Assignee: Honeywell International Inc., Morristown, NJ (US)

(21) Appl. No.: 10/660,354

(22) Filed: Sep. 10, 2003

(51) Int. Cl. A61L 9/16

(52) U.S. Cl. 422/4; 422/122; 422/121

(57) ABSTRACT

An air quality system comprising at least one air cleaner unit in communication with an interior air space. In one embodiment, an air quality system for an aircraft comprises a first set of air cleaner units in communication with a lower plenum of the aircraft recirculation system and a second set of air cleaner units in communication with an upper plenum of the aircraft recirculation system. Each air cleaner unit comprises at least one adsorbent unit in combination with at least one photocatalytic oxidation unit. In one embodiment, an air cleaner unit comprises a relatively weak adsorbent material upstream from a photocatalytic oxidation unit, and a relatively strong adsorbent material downstream from the photocatalytic oxidation unit. Methods for removing a pollutant from an air stream and for making an air cleaner unit are also disclosed.

### BACKGROUND OF THE INVENTION

The present invention generally relates to an air cleaner unit and to an air quality system for providing cleansed air to an interior air space. The present invention also relates to methods for removing a pollutant from an air stream via an air cleaner unit or an air quality system.

There is increasing interest in supplying clean, pollution-free air to interior air spaces, such as onboard commercial aircraft. To provide cleansed air to an interior air space, such as an aircraft cabin, it may be desirable to remove pollutants such as dust or other particulates, as well as volatile- or semi-volatile organic compounds (VOCs, SVOCs) from an air stream to be fed to the interior air space. It may further be desirable to remove, or inactivate (kill), microorganisms (e.g., various bacteria), and viruses, which may be human pathogens. In the case of a vehicle, such as an aircraft, such objectives must be accomplished by an air quality system within the constraints of weight and size of the system, as well as pressure drop across the system.

Conventional commercial aircraft feed air (e.g., bleed air from a gas turbine engine) to an environmental control system (ECS), and thence via one or more high efficiency particulate (HEPA) filters to an interior air space, e.g., cabin or flight deck of the aircraft. The ECS conditions the air it receives in terms of pressure, temperature, and humidity, but does not remove particulates or pollutants such as VOCs. Although HEPA filters of prior art air circulatory systems remove some particulates, they do not remove gaseous or molecular pollutants such as VOCs or SVOCs.

U.S. Pat. No. 6,358,374 B1 to Obee et al., discloses the use of an adsorbent bed to collect VOC pollutants. The apparatus is thermally cycled to periodically desorb the pollutants by heating the adsorbent bed. Movable doors within the apparatus act as valves to change the flow path of an air stream containing desorbed pollutants, such that the desorbed pollutants are cycled through a photocatalytic air purifier. The apparatus of Obee et al. is taken off-line during heating and regenerating the adsorbent bed, and is therefore periodically out of service.

Mitsubishi Paper Mills Limited markets a commercial product (Radit™) that comprises an adsorbent and a photocatalytic agent. The adsorbent and photocatalytic agent are co-impregnated on a single support of cardboard or paper, such that the support has dual functionality. Pollutants are adsorbed by the adsorbent on the support, and the adsorbed pollutant is oxidized via a photocatalytic agent, on the same support. Locating both adsorbent and photocatalytic agent on the same support has a number of disadvantages. For example, certain adsorbents may be damaged and inactivated by exposure to the UV light used for oxidizing pollutants via the photocatalytic agent. In addition, the cardboard or paper support may also be damaged by exposure to the UV light. Such damage to the support may result in the loss of impregnated adsorbent and/or photocatalytic agent over time.

Additionally, placing both adsorbent and photocatalytic agent on the same support limits the loading of each since they compete with each other for locations on the support. Furthermore, putting the adsorbent and the photocatalytic agent on the same support means that both are in powder form, which limits the adsorbent capacity for unit volume and weight as compared with self-supporting woven or pleated adsorbent media. Still further, putting adsorbent and photocatalytic agent on the same support limits flexibility in configuring an air quality system for different situations or environments.

As can be seen, there is a need for an air cleaner unit that can be used continuously over an extended period of time without the need to regenerate a bed of adsorbent. There is a further need for an air cleaner unit that may be operated at a substantially constant temperature from about 15 to 30° C., i.e., does not require thermal cycling or a heating step. There is another need for an air cleaner unit that does not require moving parts to change the flow path of an air stream, but which instead provides a single flow path for an air stream. There is a still further need for an effective air cleaner unit that includes a photocatalytic support material that is not damaged by exposure to UV light. There is an additional need for an air cleaner unit in which an adsorbent unit is physically separated from a photocatalytic oxidation unit, such that an adsorbent material of the adsorbent unit is not damaged by UV light from the photocatalytic oxidation unit.

There is a further need for an air quality system for an aircraft that not only removes particulates, but also inactivates viruses, bacteria, and other microorganisms, as well as removing pollutants such as VOCs. The present invention provides such air cleaner units, air quality systems, and methods, as will be described in enabling detail hereinbelow.

### SUMMARY OF THE INVENTION

In one aspect of the present invention, there is provided an air quality system for removing a pollutant from an air stream, the air quality system including an interior air space, and at least one air cleaner unit in communication with the interior air space, wherein the at least one air cleaner unit provides a single flow path for the air stream. The at least one air cleaner unit comprises a first photocatalytic oxidation unit and a first adsorbent unit, and the first photocatalytic oxidation unit is located upstream or downstream from the first adsorbent unit.

In another aspect of the present invention, there is provided an air quality system for an aircraft, including an interior air space including a cabin, a lower plenum, and an upper plenum; a mix manifold for distributing air to the interior air space; a lower recirculation system in communication with the mix manifold; an upper recirculation system in communication with the lower recirculation system; at least one air intake unit in communication with the mix manifold; and a plurality of air cleaner units in communication with the interior air space. Each of the plurality of air cleaner units includes at least one photocatalytic oxidation unit and at least one adsorbent unit, wherein the at least one photocatalytic oxidation unit is located upstream or downstream from the at least one adsorbent unit.

In still another aspect of the present invention, there is provided a vehicle, including an air quality system. The air quality system includes at least one air cleaner unit and an interior air space. The at least one air cleaner unit is in communication with the interior air space, wherein the at least one air cleaner unit comprises a first photocatalytic oxidation unit, a first adsorbent unit, and a second adsorbent unit, the first photocatalytic oxidation unit is located downstream from the first adsorbent unit, and the second adsorbent unit is located downstream from the first photocatalytic oxidation unit.

In yet another aspect of the present invention, there is provided an air cleaner unit for removing a pollutant from an air stream, the air cleaner unit including a housing; a first photocatalytic oxidation unit arranged within the housing; and a first adsorbent unit arranged parallel to the first photocatalytic oxidation unit in the housing, wherein the housing defines a single flow path for the air stream.

In a further aspect of the present invention, there is provided an air cleaner unit for removing a pollutant from an air stream, the air cleaner unit including a first adsorbent unit; a first photocatalytic oxidation unit located downstream from the first adsorbent unit; a second adsorbent unit located downstream from the first photocatalytic oxidation unit; and a housing. The first adsorbent unit, the first photocatalytic oxidation unit, and the second adsorbent unit are arranged parallel to each other within the housing, wherein the housing defines a single flow path for the air stream. The first adsorbent unit, the first photocatalytic oxidation unit, and the second adsorbent unit are arranged orthogonal to the air stream, wherein the first photocatalytic oxidation unit comprises a plurality of photocatalytic panels and a plurality of UV lamps so disposed as to illuminate the photocatalytic panels, each of the plurality of photocatalytic panels including a photocatalytic support and a photocatalytic agent disposed on the photocatalytic support.

In an additional aspect of the present invention, there is provided a method for removing a pollutant from an air stream, the method including providing at least one air cleaner unit, the air cleaner unit including a first adsorbent unit, a first photocatalytic oxidation unit, and a second adsorbent unit, wherein the first photocatalytic oxidation unit is located downstream from the first adsorbent unit and the second adsorbent unit is located downstream from the first photocatalytic oxidation unit; passing the air stream through the first adsorbent unit, the first adsorbent unit including a first adsorbent material having a first isotherm curve for adsorption of the pollutant; thereafter, passing the air stream through the first photocatalytic oxidation unit; and thereafter, passing the air stream through the second adsorbent unit, the second adsorbent unit including a second adsorbent material having a second isotherm curve for adsorption of the pollutant.

In another aspect of the present invention, there is provided a method for making an air cleaner unit for removing pollutants from an air stream, the method including providing a first photocatalytic oxidation unit; providing a first adsorbent unit; providing a housing for accommodating the first photocatalytic oxidation unit and the first adsorbent unit; arranging the first adsorbent unit in the housing; and arranging the first photocatalytic oxidation unit in the housing such that the first photocatalytic oxidation unit is located downstream or upstream from the first adsorbent unit, wherein the housing is adapted for providing a single flow path for passage of the air stream through the first photocatalytic oxidation unit and the first adsorbent unit.

These and other features, aspects and advantages of the present invention will become better understood with reference to the following drawings, description and claims.

### BRIEF DESCRIPTION OF THE DRAWINGS

FIG. 1 is a block diagram schematically representing a vehicle including an air quality system, according to one embodiment of the invention;

FIGS. 2A-C are block diagrams, each schematically representing an air quality system, according to the invention;

FIG. 3 schematically represents an air quality system, according to another embodiment of the invention;

FIG. 4 schematically represents an air quality system for an aircraft, according to another embodiment of the invention;

FIG. 5 schematically represents an air cleaner unit, according to one aspect of the invention;

FIGS. 6A-C schematically represent various configurations of an air cleaner unit, according to the invention;

FIG. 7A is a side view schematically representing a photocatalytic oxidation unit for an air cleaner unit;

FIG. 7B is a face view of the photocatalytic oxidation unit of FIG. 7A taken along the lines 7B-7B;

FIG. 7C is a cross-sectional view of a photocatalytic panel of a photocatalytic oxidation unit, according to one aspect of the invention;

FIG. 8 schematically represents a series of steps involved in a method for removing a pollutant from an air stream, according to another embodiment of the invention;

FIG. 9 schematically represents a series of steps involved in a method for making an air cleaner unit, according to another embodiment of the invention;

FIG. 10 is a plot of fractional removal of a pollutant per photocatalytic panel of a photocatalytic oxidation unit against inlet pollutant concentration, according to Example 3;

FIG. 11 shows a breakthrough curve for the adsorption of toluene by an activated carbon fiber adsorbent material, according to Example 4;

FIG. 12A shows a breakthrough curve for the adsorption of toluene by an activated carbon fabric adsorbent material, according to Example 5;

FIG. 12B shows the desorption of toluene by activated carbon fabric adsorbent material, also according to Example 5; and

FIG. 12C shows the rates of toluene adsorption and of toluene desorption by activated carbon fabric adsorbent material, also according to Example 5.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention provides an air quality system and method for removing pollutants from an air stream, and for providing cleansed air to an interior air space, such as a cabin or flight deck of an aircraft. As an example, the present invention may be used to provide cleansed air to the interior air space(s) of a commercial aircraft. An air quality system and method of the invention may also be applicable to other vehicles, or to buildings, in which it is desired, or required, to remove pollutants from an interior air space, or from an air stream to be delivered to the interior air space.

Air provided to an interior air space of an aircraft may contain VOCs or other pollutants at levels well above the threshold level at which they can be detected by the human olfactory system. Such pollutants may originate from within the aircraft (e.g., the cabin), or from outside the aircraft (e.g., ground vehicle exhaust gases), and may be discharged into the interior air space of the aircraft as pulses of fluctuating concentration.

In prior art air distribution systems of conventional commercial aircraft, HEPA filters do not remove pollutants, such as VOCs, or potentially harmful microorganisms and viruses.

In contrast to the prior art, air quality systems of the invention combine a particulate filter (e.g., HEPA filter), together with at least one discrete adsorbent unit and at least one discrete photocatalytic oxidation unit, to not only remove particulates from an air stream, but also to remove pollutants such as VOCs, and to remove or inactivate pathogenic microorganisms, and the like. Each of the at least one adsorbent unit and the at least one photocatalytic oxidation unit of the invention may be a discrete unit which is physically separated from other units or components of the system. As used herein, the term VOC (volatile organic compound) may include semi-volatile organic compounds (SVOCs).

Furthermore, and unlike the prior art, air cleaner units of the present invention can be used continuously over an extended period of time, for example, for a period of several years, without the need to regenerate an adsorbent material or adsorbent unit of such air cleaner units. Still further, air cleaner units of the present invention can be operated at a substantially constant temperature, e.g., at or close to ambient temperature, such as in the range of from about 15 to 30° C. In addition, air cleaner units of the present invention do not require moving parts to change the flow path of an air stream, but instead may provide a single flow path for an air stream from which a pollutant is to be removed. Further, and still in contrast to the prior art, air cleaner units of the invention include a photocatalytic support material that is not damaged by exposure to ultraviolet (UV) light.

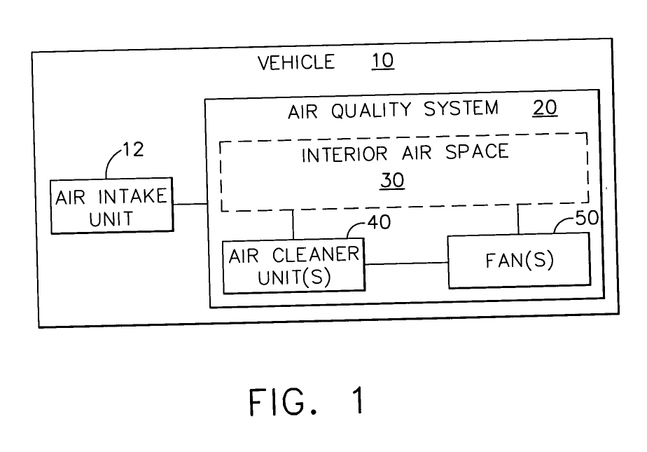

FIG. 1 is a block diagram schematically representing a vehicle 10 which may include an air quality system 20 in communication with an air intake unit 12. Air quality system 20 may include one or more air cleaner units 40 in communication with an interior air space 30. Air quality system 20 may further include one or more fans 50 in communication with the one or more air cleaner units 40 and interior air space 30. Fan(s) 50 may provide air flow within ducts (not shown in FIG. 1) and through air cleaner unit(s) 40 of air quality system 20.

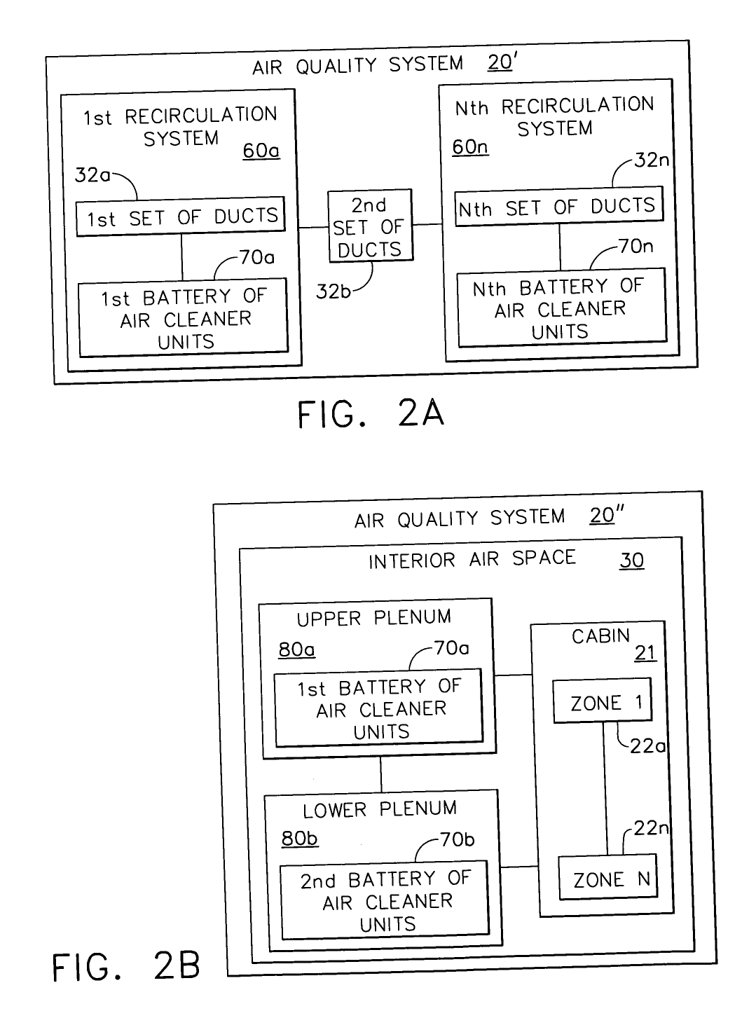

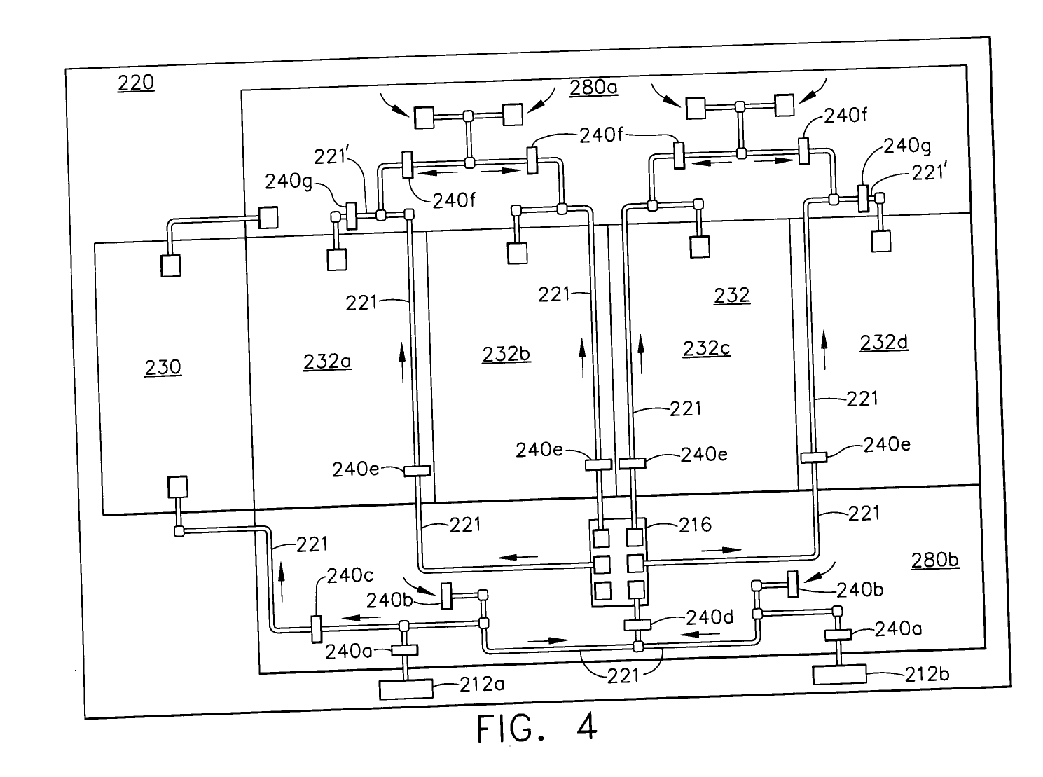

FIG. 2A is a block diagram schematically representing an air quality system 20′, according to the invention. Air quality system 20′ may include at least some of the elements and characteristics of air quality system 20 as described hereinabove with reference to FIG. 1. Air quality system 20′ may further include additional elements not shown in FIG. 1. For example, air quality system 20′ may include a first recirculation system 60a. First recirculation system 60a may include a first battery of air cleaner units 70a that may be in communication with, and/or coupled to, a first set of ducts 32a. The expression “battery of air cleaner units” may be used to denote a set of one or more air cleaner units (see, e.g., FIG. 4) that have a similar function, or an equivalent location with respect to certain other components of an air quality system of the invention. Examples of equivalent locations for members of a battery of air cleaner units are shown in FIG. 4.

First recirculation system 60a may be coupled to one or more additional recirculation systems via a second set of ducts 32b. Such one or more additional recirculation systems are represented in FIG. 2A as N^{th} recirculation system 60n. N^{th} recirculation system 60n may include an N^{th} battery of air cleaner units 70n in communication with, and/or coupled to, an Nth set of ducts 32n. Air quality system 20′ may also include the same, or similar, features as described for air quality system 20 of FIG. 1.

FIG. 2B is a block diagram schematically representing an air quality system 20″ including an interior air space 30, according to another embodiment of the invention. Interior air space 30 may include an upper plenum 80a in communication with a cabin 21. Interior air space 30 may further include a lower plenum 80b in communication with both upper plenum 80a and cabin 21. As an example only, and not to limit the invention, cabin 21 may be in communication with both upper plenum 80a and lower plenum 80b via one or more vents, while upper plenum 80a may be in communication with lower plenum 80b via one or more ducts (not shown in FIG. 2B). Vents and ducts within air spaces are well known in the art. According to one aspect of the invention, upper plenum 80a may house first recirculation system 60a (FIG. 2A), while lower plenum 80b may house a further recirculation system (e.g., Nth recirculation system 60n (FIG. 2A)).

Again with reference to FIG. 2B, cabin 21 may include a plurality of zones, represented in FIG. 2B as zone 1 (22a) and zone N (22n). Air quality system 20″ may further include a first battery of air cleaner units 70a and a second battery of air cleaner units 70b, which may be housed within upper plenum 80a and lower plenum 80b, respectively.

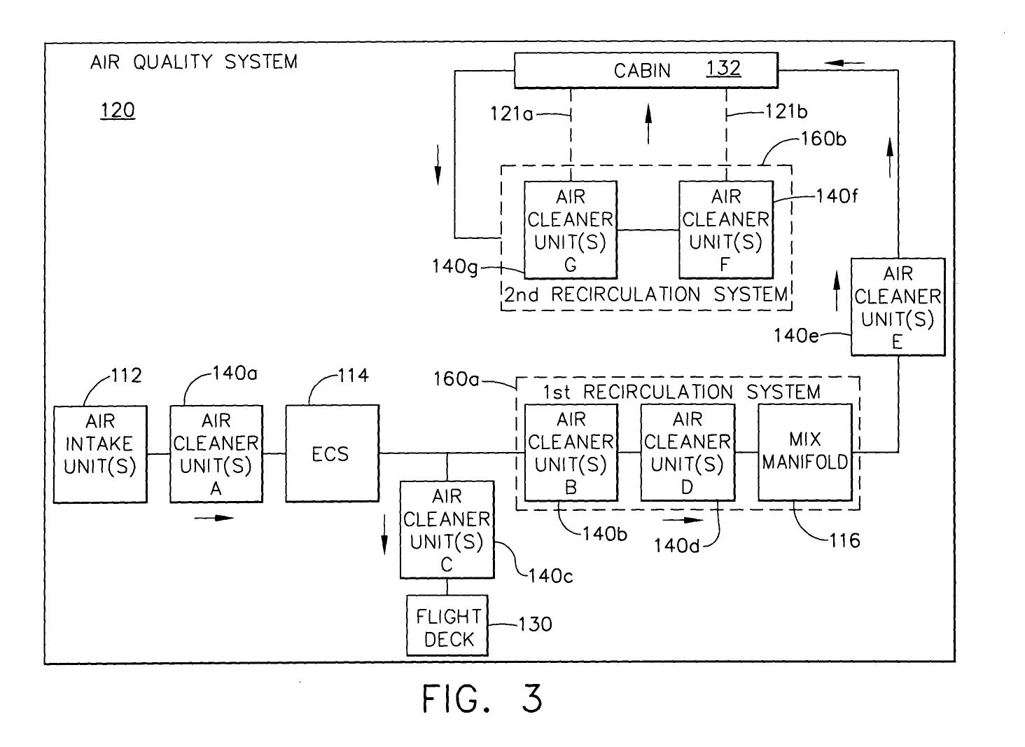

FIG. 3 schematically represents an air quality system 120, according to another embodiment of the invention. FIG. 3 shows an example of how a plurality of air cleaner units, or a plurality of batteries of air cleaner units, may be arranged with respect to each other, and with respect to other elements (e.g., one or more recirculation systems) of an air quality system of the invention. In one aspect, a function of air quality system 120 is to provide cleansed air to an interior air space. In another aspect, cleansed air may be provided to an interior air space by removing one or more pollutants from an air stream containing the one or more pollutants. Air quality system 120 can be described, at least in part, with reference to features of an aircraft, it being understood that the elements, characteristics, and functions of air quality system 120 may be equally applicable to other vehicles, buildings, and the like, having an interior air space.

Air quality system 120 may include a plurality of air cleaner units, which for convenience may be designated as air cleaner unit(s) A-G in FIG. 3. Air cleaner unit(s) A-G are assigned the reference numerals 140a-g, respectively. Each of air cleaner unit(s) 140a-g may comprise a single air cleaner unit or a set, or battery, of air cleaner units. Typically, each air cleaner unit of a battery of air cleaner units may have an equivalent location with respect to certain other components of air quality system 120, and an equivalent function within air quality system 120. Air cleaner unit(s) 140a-g may be coupled to each other, or to other elements of air quality system 120, via one or more ducts (not shown in FIG. 3) for channeling air therethrough. Such ducts are well known in the art.

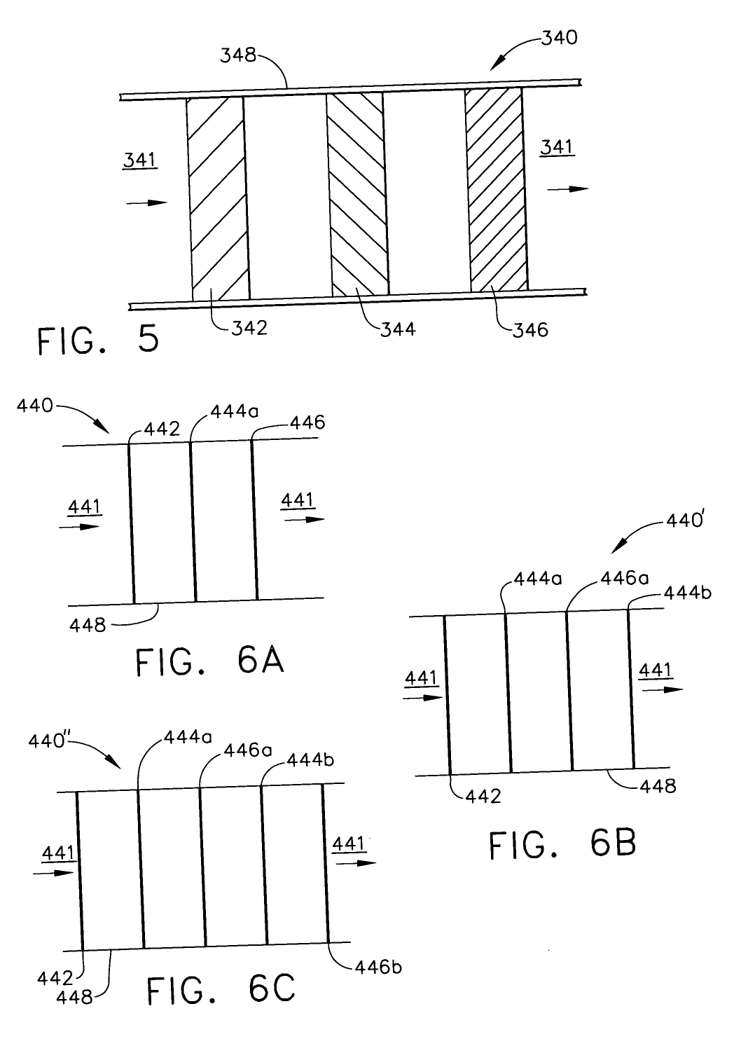

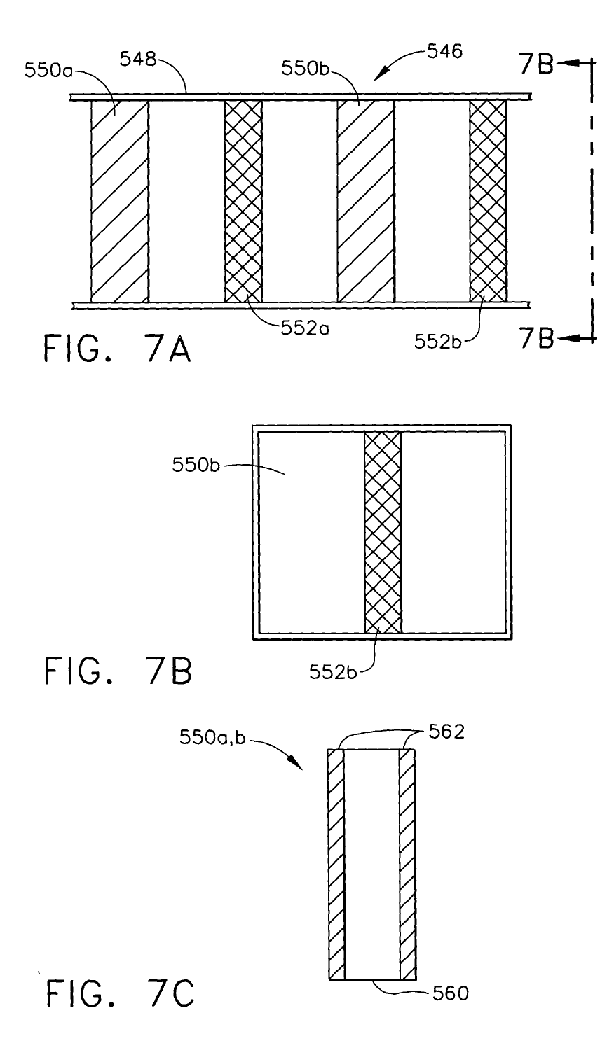

It should be understood that air quality system 120 need not include all of air cleaner unit(s) 140a-g, but instead various embodiments of air quality system 120 may include one, or a combination of two or more, air cleaner unit(s) 140a-g. Each of air cleaner unit(s) 140a-g may comprise one or more photocatalytic oxidation (PCO) units (e.g., PCO units 446a,b, FIG. 6C) located upstream or downstream from one or more adsorbent units (e.g., adsorbent units 444a,b, FIG. 6C). The structure and function of air cleaner unit(s) 140a-g are described fully hereinbelow (e.g., with reference to FIGS. 5, 6A-C).

Air quality system 120 may further include one or more air intake units 112, e.g., for the intake of air from outside an aircraft cabin. As an example, air intakes units 112 may be in the form of ducts leading from gas turbine engine(s) of the aircraft for carrying bleed air from the engines towards an environmental control system (ECS) 114. Such outside air may be directed as an air stream to air cleaner unit(s) 140a. Closed arrows indicate the direction of the air stream, or the direction of air flow, within air quality system 120. Air flowing from air cleaner unit(s) 140a may be passed to ECS 114. (ECS of aircraft are well known in the art. Briefly, the ECS of an aircraft controls the pressure, temperature and humidity of air received by the ECS.) A stream of air may be fed from ECS 114 to a mix manifold 116, and thence to an interior air space, which typically includes a cabin of the aircraft. It is apparent from FIG. 3 that air cleaner unit(s) 140a may be located upstream from ECS 114. The remaining air cleaner unit(s) 140b-g shown in FIG. 3 may be located downstream from ECS 114. From ECS 114, the air stream may be directed through air cleaner unit(s) 140b. Thereafter, the air stream may be directed to air cleaner unit(s) 140d, which is located downstream from air cleaner unit(s) 140b.

Mix manifold 116 may be located downstream from air cleaner unit(s) 140d. Air quality system 120 may further include a first recirculation system 160a. Mix manifold 116, together with air cleaner unit(s) 140b and 140d, may be arranged within first recirculation system 160a. First recirculation system 160a may recirculate air within an interior air space (e.g., FIGS. 1, 2B, 4).

Air quality system 120 may still further include a flight deck 130. Air cleaner unit(s) 140c may be arranged between flight deck 130 and ECS 114, to provide cleansed air to flight deck 130. Air quality system 120 may still further include a second recirculation system 160b. First and second recirculation systems 160a,b may each comprise a series of ducts, together with one or more fans coupled to the ducts for drawing air into the ducts. In a commercial aircraft, first and second recirculation systems 160a,b may be housed within the lower plenum and upper plenum, respectively (see, e.g., FIG. 4).

Air cleaner unit(s) 140e may be arranged between first recirculation system 160a and cabin 132. Cabin 132 may be in communication with second recirculation system 160b. Air cleaner unit(s) 140g, 140f may be arranged within second recirculation system 160b. Cabin 132 may be coupled to air cleaner unit(s) 140g, 140f via cabin supply lines 121a and 121b, respectively, through which air passed from air cleaner unit(s) 140g, 140f may be delivered to cabin 132. Each of air cleaner units 140a-g can be adapted for removing pollutants from an air stream passed therethrough, such that a supply of cleansed air may be delivered to flight deck 130 and/or cabin 132.

FIG. 4 schematically represents an air quality system 220 for an aircraft, according to another embodiment of the invention. Air quality system 220 may include a flight deck 230, a cabin 232, an upper plenum 280a, and a lower plenum 280b. Flight deck 230, cabin 232, upper plenum 280a, and lower plenum 280b may define all or a portion of an interior air space to which air quality system 220 may provide a supply of cleansed air by removing one or more pollutants from an air stream. The air stream may emanate, in part, from air intake units 212a-b, and be passed throughout air quality system 220 via a series of ducts 221.

Cabin 232 may include a plurality of zones, for example, first, second, third, and fourth zones 232a-d, respectively. Cabin 232 may be in communication with upper plenum 280a and lower plenum 280b via one or more vents (not shown). Such vents are well known in the art.

Air quality system 220 may further include one or more air cleaner units, for example, air cleaner units 240a-g. Each of air cleaner units 240a-g can be adapted for removing one or more pollutants from the air stream.

Each of air cleaner units 240a-g may comprise a single air cleaner unit or a plurality of air cleaner units in the form of a battery of air cleaner units. Typically, each member of a battery of air cleaner units may perform an equivalent function, and may occupy an equivalent location, within air quality system 220. As an example, air cleaner units 240a may comprise a pair of air cleaner units located downstream from air intake units 212a-b, and upstream from an ECS (not shown in FIG. 4) of the aircraft. As another example, air cleaner units 240b may comprise a pair of air cleaner units located within lower plenum 280b. The various locations of air cleaner units 240a-g shown in FIG. 4 are described in Table 1. The lower recirculation system generally comprises those ducts 221 housed within lower plenum 280b, together with one or more fans (FIG. 1) coupled to ducts 221. The upper recirculation system generally comprises those ducts 221 housed within upper plenum 280a.

TABLE 1
Air Cleaner Unit Locations of FIG. 4

| Air Cleaner | |
|---|---|
| Unit No. | Filter Location Description |
| 240a | Air Intake Lines |
| 240b | Lower Recirculation System |
| 240c | To Flight Deck |
| 240d | To Mix Manifold |
| 240e | From Mix Manifold |
| 240f | Upper Recirculation System |
| 240g | Cabin Supply Lines |

Air received by air cleaner units 240b from lower plenum 280b may be combined with air emanating from air intake units 212a-b, and passed to mix manifold 216. Air en route to mix manifold 216 may be passed through air cleaner unit 240d, which is located upstream from mix manifold 216. Air may then be distributed from mix manifold 216 via ducts 221 towards first, second, third, and fourth zones 232a-d, respectively, of cabin 232. Such air distributed from mix manifold 216 may be passed through air cleaner units 240e. Air received from upper plenum 280a, and passing through air cleaner units 240f, may be cleansed before being mixed with air emanating from mix manifold 216. Thereafter, the mixed air may be passed to cabin supply lines 221′. Air passing through cabin supply lines 221′ may be passed through air cleaner units 240g before being discharged into cabin 232.

Air cleaner units 240a-g may each comprise one or more PCO units in combination with one or more adsorbent units, as described hereinbelow, e.g., with reference to FIGS. 5, 6A-C. The one or more PCO units and one or more adsorbent units may be arranged within ducts 221, such that ducts 221 may provide a housing for the one or more PCO units and one or more adsorbent units. In alternative embodiments, one or more of air cleaner units 240a-g may have a separate housing, which may be coupled to ducts 221.

It is to be understood that FIG. 4 indicates a number of possible locations for air cleaner units 240a-g. For example, various embodiments of air quality system 220 may include one, or a combination of two or more, air cleaner unit(s) 240a-g. As an example, in one embodiment, air quality system 220 may include air cleaner unit(s) 240b and 240c. In another embodiment, air quality system 220 may include air cleaner unit(s) 240b, 240c and 240f. In yet another embodiment, air quality system 220 may include air cleaner unit(s) 240b, 240c, 240d and 240f. In still another embodiment, air quality system 220 may include air cleaner unit(s) 240b, 240c, 240d, 240e and 240f. In yet another embodiment, air quality system 220 may include air cleaner unit(s) 240b, 240c, 240d, 240e, 240f and 240g. In still another embodiment, air quality system 220 may include air cleaner unit(s) 240b, 240c, 240d, 240e, 240f and 240g. In another embodiment, air quality system 220 may include air cleaner unit(s) 240a, 240b, 240c, 240d, 240e, 240f and 240g. Locations other than those listed in Table 1 and shown in FIG. 4 for air cleaner units 240a-g are also possible under the invention.

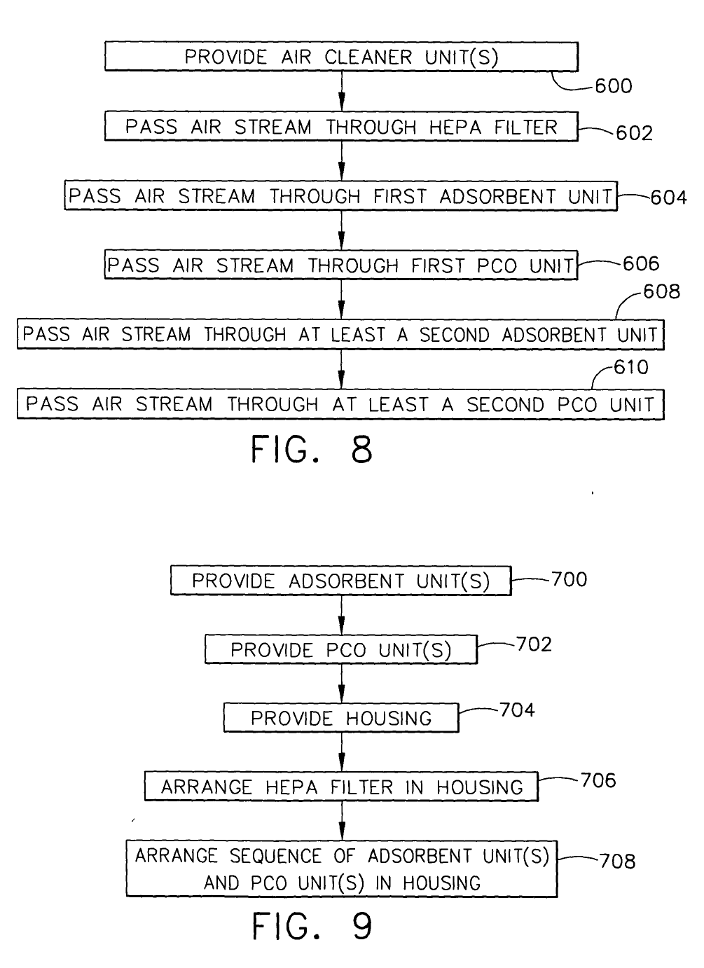

FIG. 5 schematically represents an air cleaner unit 340, according to one aspect of the instant invention. The characteristics and features described below for air cleaner unit 340 may apply equally to air cleaner units 40, 140a-g, and 240a-g (FIGS. 1, 3, and 4, respectively). Air cleaner unit 340 may include a particulate filter 342, an adsorbent unit 344 and a PCO unit 346. Each PCO unit 346 may comprise one or more PCO panels and one or more UV sources, e.g., as described hereinbelow with reference to FIGS. 7A-C. Particulate filter 342 may comprise a high efficiency particulate (HEPA) filter. Such filters are well known in the art. Particulate filter 342 may be located upstream from adsorbent unit 344 and PCO unit 346. In an alternative embodiment (not shown), an air stream 341 (represented by solid arrows) provided to air cleaner unit 340 may be filtered upstream from air cleaner unit 340 to remove at least some particulates from air stream 341, in which case particulate filter 342 may be omitted from air cleaner unit 340.

Air cleaner unit 340 may further include a housing 348. Particulate filter 342, adsorbent unit 344, and PCO unit 346 may be arranged parallel, or substantially parallel, to each other, and orthogonal, or substantially orthogonal, to the direction of air steam 341, within housing 348. As an example, housing 348 may comprise a duct for the passage of air within a vehicle, such as an aircraft, or building. At least in the vicinity of adsorbent unit 344 and PCO unit 346, housing 348 may be linear or follow a straight line Housing 348 may be adapted for the unidirectional passage of air stream 341. Particulate filter 342, adsorbent unit 344, and PCO unit 346 may each extend to the entire perimeter of housing 348.

Housing 348 may provide only a single flow path for air stream 341 throughout the entire extent, or length, of air cleaner unit 340. Air cleaner unit 340 does not require the use of moving parts, such as doors or valves, for redirecting the flow of an air stream 341 through components of air cleaner unit 340. Consequently, air stream 341 may follow only a single flow path, and the flow of air in air stream 341 may be unidirectional. Similarly, operation of air cleaner unit 340 does not require the taking of one or more components of air cleaner unit 340 off-line, e.g., for regeneration of adsorbent unit 344. Instead, air cleaner unit 340 has the advantage of being adapted to provide a continuous supply of cleansed air to an interior space.

In FIG. 5, adsorbent unit 344 is shown as being located upstream from PCO unit 344. However, adsorbent unit 344 may be located upstream or downstream from PCO unit 346. Also, according to various embodiments of the invention, one or more adsorbent units 344 may be arranged in various sequences with one or more PCO units 346 (see, e.g., FIGS. 6A-C).

Each adsorbent unit 344 may include an adsorbent material capable of adsorbing one or more pollutants, such as a volatile- or semi-volatile organic carbon (VOC, SVOC), or a gas, such as ammonia, methane, carbon monoxide, hydrogen sulfide, and the like. VOCs, such as various hydrocarbons, aldehydes, aromatics, toluene, etc., are well known in the art as pollutants within interior air spaces. Such VOC's may be odorous and create an uncomfortable environment for passengers and crew. Various pollutants may be of biological origin, may be derived from ingested materials, may be emitted from chemical processes, or may be discharged from motors or engines in the vicinity of an interior air space. An adsorbent material of adsorbent unit(s) 344 may comprise, for example, various forms of activated carbon, silica gel, or a zeolite. In general, such adsorbents are well known in the art.

According to one aspect of the invention, an adsorbent material of adsorbent unit(s) 344 may comprise a fine porosity adsorbent having a plurality of macropores, each of the plurality of macropores in communication with at least one micropore. Each micropore may have a diameter in the range of from about 5 to 10 Å. Typically, at least about 30% of the micropores may have a diameter in the range of from about 5 to 10 Å, and usually at least about 50% of the micropores may have a diameter in the range of from about 5 to 10 Å.

Each PCO unit 346 may include one or more photocatalytic (PC) panels in combination with one or more UV sources (e.g., a UV lamp or other source of ultra-violet radiation), as described below in connection with, for example, FIGS. 7A-C. Each PCO unit 346 may photocatalytically destroy or remove, e.g., via oxidation to carbon dioxide and water, a pollutant such as a VOC from air stream 341. Thus, PCO unit(s) 346 may act in concert with adsorbent unit(s) 344 to remove one or more pollutants from air stream 341, as is described fully hereinbelow. In addition, PCO unit(s) 346 may destroy biological agents, e.g., viruses, bacteria, and other air-borne microorganisms, as well as pollen, fungal spores, or other allergens. Bacteria and viruses may be inactivated by exposure to UV radiation from the UV source alone, or by exposure to the UV source while in the presence of a PC agent of each PC panel. Such viruses, bacteria, and other microorganisms may include human pathogens.

FIGS. 6A-C schematically represent different configurations for an air cleaner unit, according to various embodiments of the invention. Air cleaner units 440, 440′, and 440″ shown in FIGS. 6A, 6B and 6C, respectively, may have the same or similar features to those of FIG. 5. With reference to FIG. 6A, air cleaner unit 440 may include a housing 448 and a particulate filter 442 arranged within housing 448. Housing 448 may provide a single flow path for air stream 441 flowing within housing 448 and through air cleaner unit 440. Particulate filter 442 may be arranged substantially orthogonal to the direction of air stream 441 within housing 448.

Air cleaner unit 440 may further include a first adsorbent unit 444a and a first PCO unit 446a arranged within housing 448. First adsorbent unit 444a and first PCO unit 446a may be arranged substantially parallel to each other and substantially parallel to particulate filter 442. First adsorbent unit 444a may be located upstream from first PCO unit 446a and may be located downstream from particulate filter 442.

With reference to FIG. 6B, air cleaner unit 440′ may include those elements described hereinabove for air cleaner unit 440 (FIG. 6A). Air cleaner unit 440′ may additionally include a second adsorbent unit 444b. Second adsorbent unit 444b may be located downstream from first PCO unit 446a. First adsorbent unit 444a may include a first adsorbent material, while second adsorbent unit 444b may include a second adsorbent material. As an example, the first and second adsorbent materials may each be selected from a silica gel, various carbonaceous materials, such as an activated carbon or a carbon fabric, and a zeolite. In one embodiment, the second adsorbent material may have a steeper isotherm curve for a pollutant adsorbate, as compared with that of the first adsorbent material.

With reference to FIG. 6C, air cleaner unit 440″ may include those elements described hereinabove for air cleaner units 440, 440′ (FIG. 6B). Air cleaner unit 440″ may additionally include a second PCO unit 446b. Second PCO unit 446b may be located downstream from second adsorbent unit 444b.

Air cleaner units of the invention, e.g., air cleaner units 440′, 440″, having a first adsorbent material upstream from first PCO unit 444a, when combined with a second adsorbent material located downstream from first PCO unit 444a, may provide unique characteristics and advantages to such air cleaner units 440′, 440″.

While not being bound by theory, the advantages associated with using particular combinations or sequences of adsorbent units and PCO units may be better understood by considering the thermodynamics behind the adsorption process. When an adsorbent material is in contact with a given concentration of pollutant (adsorbate), an equilibrium is established between adsorbed and vapor phase concentrations of pollutant, wherein the equilibrium is a property of the adsorbent and adsorbate. When the pollutant concentration increases, the quantity of pollutant adsorbed will also increase. However, when the vapor phase concentration of the pollutant subsequently decreases, a portion of the sorbed pollutant will tend to desorb. This situation may apply, for example to a first adsorbent unit located upstream from any other adsorbent unit or PCO unit of an air cleaner unit. Pollutant desorbed from the first adsorbent unit may be removed by a first PCO unit downstream from the first (upstream) adsorbent unit. In this way, any desorbed pollutant may be prevented from being discharged into an interior air space.

For example, a pulse in pollutant concentration in an air stream entering an air cleaner unit of the invention will adsorb onto a first adsorbent unit. The higher pollutant concentration in the air stream thermodynamically translates into a higher adsorption capacity for the adsorbent of the first adsorbent unit. Consequently, the PCO unit downstream from the first adsorbent unit may be spared from receiving an unmanageably high pulse or concentration of the pollutant. After the pulse of pollutant has passed, the equilibrium of the adsorbent of the first adsorbent unit is restored by desorption of the pollutant which was adsorbed during the pulse. Such pollutant desorbed by the first adsorbent unit passes to the downstream PCO unit where it may be destroyed by oxidation. The first adsorbent unit thus serves to store a pulse of elevated pollutant concentration, and subsequently meters the pollutant (via equilibrium-driven desorption) to the PCO unit at a manageable rate. The capacity of the first adsorbent unit is not consumed, since the extra adsorbent sites used to adsorb the pulse of pollutant do so reversibly.

The operation of air cleaner units of the invention may be further improved by including at least one additional adsorbent unit, e.g., a second adsorbent unit, downstream from the at least a first PCO unit. As observed hereinabove, a second adsorbent unit may include a second adsorbent material having adsorption properties different from those of a first adsorbent material of a first adsorbent unit, e.g., the first and second adsorbent materials may have a different isotherm curve. For an adsorbent unit which is located downstream from all PCO units of a given air cleaner unit of the invention, an adsorbent material having a relatively steep isotherm curve may be used. This is because adsorbent materials having steeper isotherm curves will tend to desorb pollutants only very slowly, or adsorb pollutants irreversibly, thereby preventing or minimizing the release of such pollutants. The quantity of adsorbent needed for the second adsorbent unit may be much less than would otherwise be required in the absence of an upstream PCO unit, since the PCO unit consumes most of the impurity. In some embodiments, the second adsorbent unit may function as a “guard bed,” by preventing pollutant which would otherwise escape the system from doing so. The second adsorbent unit may also function to remove any partially oxidized pollutant degradation products, which might result from incomplete oxidation of pollutant by the PCO unit.

For an adsorbent which is placed upstream from one or more PCO units, an adsorbent material having a less steep isotherm curve may be used. An adsorbent material which is “weaker” (i.e., has a less steep isotherm curve for a particular pollutant), will tend to desorb the pollutant as the pollutant concentration decreases from a transiently higher concentration, or pulse, of the pollutant. In this way, the “weaker” adsorbent material will tend to act as a “buffer,” thereby variation in pollutant concentration reaching downstream PCO units and other adsorbent units is minimized. Examples of adsorbent materials having a relatively steep isotherm curve, and which may be used in a second or downstream adsorbent unit, include certain forms of carbon and zeolites. Examples of adsorbent materials having a less steep isotherm curve, and which may be used in a first or upstream adsorbent unit, include silica gel and some carbon materials.

Isotherm curves, which show the distribution of adsorbate (e.g., a pollutant to be adsorbed) between the adsorbed phase (adsorbed on an adsorbent material) and the vapor phase at equilibrium, are well known in the art (see, for example, Encyclopedia of Separations Technologies (D. M. Ruthven ed.), Adsorption Equilibrium Data Handbook (Valenzuela & Meyers, 1998); Carbon Adsorption Isotherms for Toxic Organics (Dobbs & Cohen, 1980), which are incorporated herein by reference).

Selecting an appropriate adsorbent material for an adsorbent unit of the invention may be, at least to some extent, a matter of design choice. The choice of adsorbent material may depend on, for example, the operating conditions of an air filter unit of which the adsorbent unit is a part, as well as on the nature of other components of the air filter unit, e.g., the nature of the PCO unit(s). For example, if during operation, it is expected that there will frequently be a relatively high concentration (e.g., >10 ppm) of pollutant for a relatively extended period (e.g., >15 minutes), followed by an extended period (e.g., >15 minutes) of no load, an adsorbent having a relatively large pore capacity (e.g., >1000 m²/g) and broad porosity (e.g., having pore diameters in the range of from about 6 to 600 Å) may be useful. However, for the removal of pollutants present at generally low concentration (e.g., <10 ppm) with periodic spikes (<15 minutes), adsorbents having a relatively high surface area (e.g., having a surface area >1000 m²/g, usually from about 1000-2500 m²/g, and often from about 1500-2500 m²/g) and having fine porosity (e.g., having micropores in the range of about 5-10 Å in diameter) may be preferred. Such fine porosity adsorbents may have a plurality of micropores with at least about 30% of the micropores having a diameter in the range of about 5-10 Å, and usually having at least about 50% of the micropores in the 5-10 Å diameter range. While not being bound by theory, micropores in the 5-10 Å diameter range may allow condensation of the pollutant within the micropores at the low partial pressures associated with low concentrations (e.g., <10 ppm) of pollutant (see, for example, R. T. Liu, Ph.D., Use of Activated Carbon Adsorbers in HVAC Applications; 6th Indoor Air Quality Conference—IAQ 93 (Operating and Maintaining Buildings for Health, Comfort, and Productivity), held November, 1993, Philadelphia, Pa.; Published: Atlanta, Ga.: ASHRAE; ISBN: 1883413133).

Adsorbent materials having a relatively high surface area and fine porosity, and which may be useful in practicing the present invention, may be carbon based (e.g., various activated carbon materials), or may be comprised of inorganic materials. Specific examples of adsorbent carbon materials are presented in Examples 4 and 5 hereinbelow. Apart from the characteristics of adsorbents which are directly related to the capacity and kinetics of pollutant adsorption, certain forms of adsorbent materials, such as activated carbon fibers or activated carbon fabrics (e.g., Kynol™ 5092-15, American Kynol, Pleasantville, N.Y.), may also offer benefits for configuration of adsorbent units, e.g., due to the structural features or mechanical properties of the adsorbent material.

FIG. 7A is a side view schematically representing a PCO unit 546. As an example, PCO unit 546 may be a component of air cleaner units 40, 240a-g, 340, or 440-440″, as described hereinabove with reference to FIGS. 1, 4, 5, and 6A-C, respectively. PCO unit 546 may include at least one PC panel and at least one UV source (or UV lamp), wherein the at least one PC panel includes a UV-absorbing PC agent (e.g., FIG. 7C). The at least one UV source may be disposed so as to illuminate the at least one PC panel. Each UV source may belong to any of the classes UV-A, UV-B or UV-C, and may be selected to emit at a wavelength at which the PC agent adsorbs. Lamps are typically long cylindrical units (e.g., FIG. 7B) or U-shaped units (not shown), with electrodes at either end. Examples of UV lamps which might be used as a UV source in PCO unit 546 include mercury arc lamps, xenon arc lamps, and the like.

PCO unit 546 may include a first PC panel 550a, a first UV source 552a, a second PC panel 550b, and a second UV source 552. PCO unit 546 may further include a housing 548. Housing 548 may comprise a duct or a portion of a duct. As an example, housing 548 may comprise a metal or a UV-resistant plastic material. First PC panel 550a, first UV source 552a, second PC panel 550b, and second UV source 552 may be arranged sequentially within housing 548 such that first and second UV sources 552a,b alternate with first and second PC panels 550a,b. In some embodiments, PCO unit 546 may include additional numbers of PC panels and additional numbers of UV sources. For example, PCO unit 546 may include up to six (6), or up to 10 or more PC panels, together with the same, or a similar, number of UV sources. The invention is not limited by the number of PC panels or by the number of UV sources.

It will be understood by those skilled in the art that conversion of pollutants at any point on a given PCO panel will be influenced by the light intensity incident on that point, such that conversions will be higher at points on the panel which are closer to a UV lamp. Air passing through a first PCO panel will have an opportunity to mix before it reaches a second PCO panel located downstream from the first PCO panel. Such mixing is beneficial, since a portion of air which may have passed through a region of the first PCO panel which was less-well illuminated may subsequently pass through a region of the second PCO panel which is better illuminated. Mixing will also occur in the spaces between the PCO units and the adsorbent units. This beneficial mixing cannot occur in prior-art designs where the adsorbent and photocatalytic material are co-located on the same structure.

FIG. 7B is a face view of PCO unit 546 of FIG. 7A taken along the lines 7B-7B, showing second UV source 552b arranged with respect to second PC panel 550b. FIG. 7C is a cross-sectional view through a PC panel 550a,b. PC panel 550a,b may include a PC support 560 and a PC agent 562 disposed on PC support 560. PC support 560 may comprise one or more sheets of a material such as a metal, which may be adapted to provide an increased surface area of PC support 560 and to facilitate air flow therethrough. In some embodiments, PC support 560 may comprise one or more sheets of expanded aluminum, which comprises a sheet of aluminum having a plurality of slits, wherein the slits are expanded in at least one direction. PC agent 562, which may comprise titanium dioxide, oxides of other metals, or cadmium sulfide, and the like, serves to photocatalytically destroy pollutants, such as VOCs, and to inactivate or kill bacteria and viruses. PC panels for use in conjunction with UV sources were described in commonly assigned U.S. patent application Ser. No. 10/345,022, filed Jan. 14, 2003, the disclosure of which is incorporated by reference herein in its entirety.

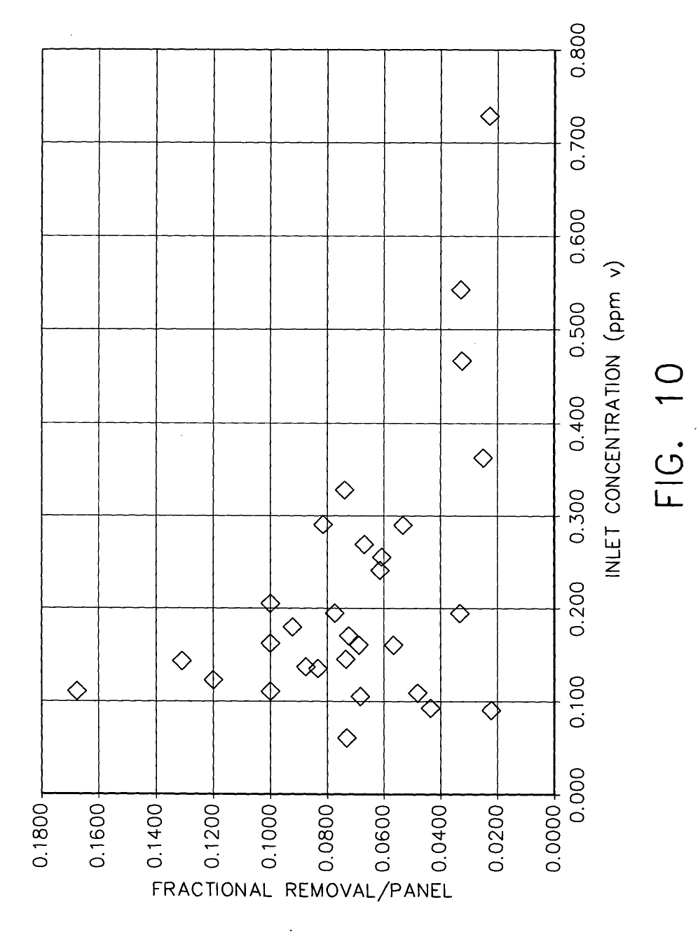

FIG. 8 schematically represents a series of steps involved in a method for removing a pollutant from an air stream, according to another embodiment of the invention. Step 600 may involve providing one or more air cleaner units. Each of the one or more air cleaner units provided in step 600 may include at least a first adsorbent unit and at least one PCO unit. Each of the one or more air cleaner units provided in step 600 may further include those elements and characteristics as described hereinabove for other embodiments of the invention, e.g., as described with reference to FIGS. 5, 6A-C.

Step 602 may involve passing an air stream through a HEPA filter, or other particulate filter, to remove at least a portion of particulate materials from the air stream. Such an air stream may be generated by recirculating air from an interior air space, e.g., via one or more fans, or by directing a source of outside air through one or more ducts, or by a combination of the above.

After step 602, step 604 may involve passing the air stream through a first adsorbent unit. In one embodiment, the first adsorbent unit includes a first adsorbent material that is adapted to adsorb and desorb a pollutant according to the relative concentrations of unadsorbed pollutant (e.g., in the vapor phase) and adsorbed pollutant (adsorbed by the adsorbent material). According to one aspect of the invention, the first adsorbent unit may serve as a “buffer” to minimize variations in concentration of pollutant in the air stream flowing downstream from the first adsorbent unit during changes in concentration of the pollutant upstream from the first adsorbent unit. The first adsorbent unit may include an adsorbent material such as a silica gel or an activated carbon in an adsorbent bed, or may include a carbon fabric adsorbent, or a carbon-coated ceramic monolith, or a solid carbon monolith, or the like. According to one aspect of the invention, the first adsorbent unit may include a fine porosity adsorbent material containing a plurality of micropores having diameters in the range of from about 5 to 10 Å. Typically, at least about 30% of the micropores may have a diameter in the range of from about 5 to 10 Å, and usually at least about 50% of the micropores may have a diameter in the range of from about 5 to 10 Å.

After step 604, step 606 may involve passing the air stream through a first PCO unit. The first PCO unit may include one or more PC panels and one or more UV sources, for example, as described hereinabove with reference to FIGS. 7A-C. The first PCO unit is adapted for photocatalytically destroying one or more pollutants in the air stream passed from the first adsorbent unit, including one or more pollutants desorbed from the first adsorbent unit, e.g., in response to a decrease in the concentration of the pollutant in the air stream reaching the first adsorbent unit.

After step 606, step 608 may involve passing the air stream through at least a second adsorbent unit. In one embodiment, the second adsorbent unit includes a second adsorbent material that is adapted to strongly, or irreversibly, adsorb a pollutant from the air stream reaching the second adsorbent unit. That is to say, the second adsorbent material may be adapted not to desorb a pollutant when the concentration of that pollutant in the vapor phase (e.g., the air stream entering the second adsorbent unit) decreases. According to one aspect of the invention, the second adsorbent unit may serve as a “trap” to indefinitely sequester the pollutant. The second adsorbent unit may include an adsorbent material such as an activated carbon, a zeolite, or the like. Impregnated adsorbents, such as an impregnated salt or cation may also be used.

As referred to hereinabove, the second adsorbent unit may be characterized as having a second adsorbent material which has a relatively steep isotherm curve, while the first adsorbent unit may be characterized as having a first adsorbent material which has a less steep isotherm curve. The total number of adsorbent units, or the nature of each of the adsorbent units, may be selected such that the adsorbent units as a whole will not be exhausted, and will not need replacement, until after a pre-defined extended period of operation. The adsorbent units and PCO unit(s) of the invention may be operated for a period of several years without maintenance.

After step 608, step 610 may involve passing the air stream through at least a second PCO unit. Second PCO unit may have characteristics and elements as described hereinabove, e.g., with reference to step 606. Because the air stream reaching the second PCO unit has already been cleansed by upstream PCO and adsorbent units, step 610 may be considered a “polishing” step. In contrast to the prior art, each of steps 602 through 610 may be performed at ambient temperature, or at a temperature close to ambient, e.g., in the range of from about 15 to 30° C.

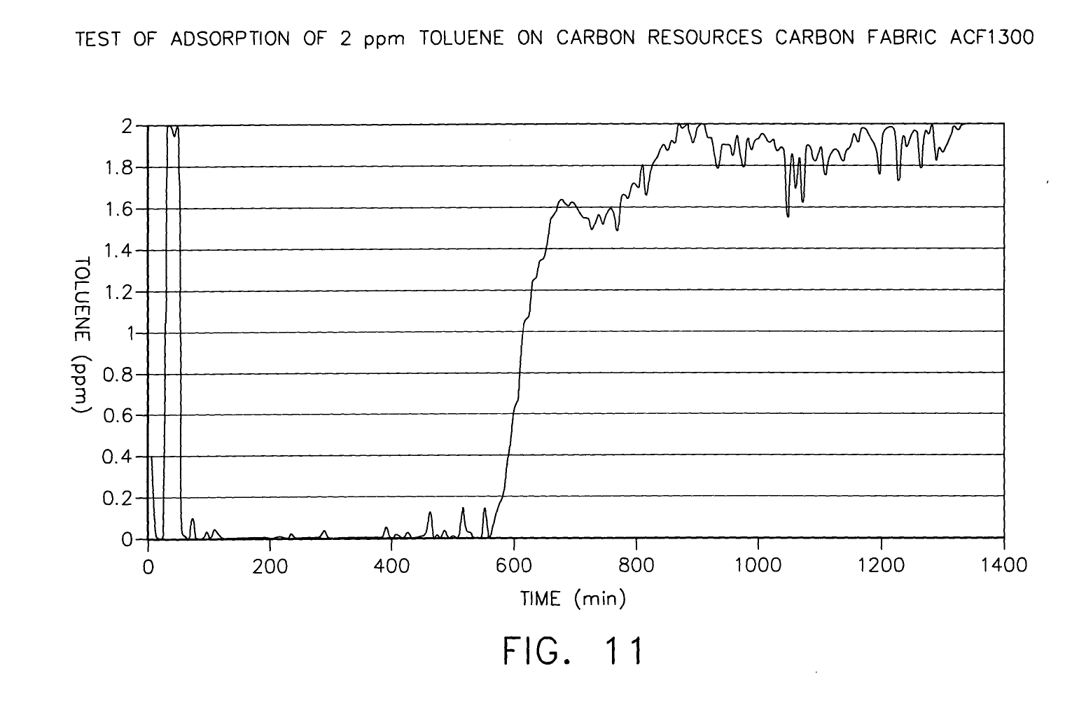

FIG. 9 schematically represents a series of steps involved in a method for making an air cleaner unit, according to another embodiment of the invention. Step 700 may involve providing one or more adsorbent units. Step 702 may involve providing one or PCO units. Each of the one or more adsorbent units and the one or PCO units provided in steps 700 and 702, respectively, may have features and elements as described hereinabove with respect to other aspects of the invention.

Step 704 may involve providing a housing. The housing provided in step 704 may house the one or more adsorbent units and the one or PCO units, and may further provide only a single flow path for the passage of an air stream through the one or more adsorbent units and the one or PCO units of the air cleaner unit. Optional step 706 may involve arranging a HEPA filter within the housing. Alternatively, in situations where a particulate filter is located upstream from the air cleaner unit, the HEPA filter may be omitted from the air cleaner unit, and step 706 may be omitted from the method of FIG. 9.

Step 708 may involve arranging the one or more adsorbent units and the one or more PCO units within the housing, such that the one or more adsorbent units and the one or more PCO units are arranged in a particular sequence with respect to each other and with respect to the HEPA filter. Typically, the HEPA filter is arranged within the housing such that the HEPA filter is upstream from both the one or more adsorbent units and the one or more PCO units. Some possible sequences for arranging the one or more adsorbent units and the one or more PCO units with respect to each other are described hereinabove, e.g., with reference to FIGS. 6A-C. Other possible sequences for arranging the one or more adsorbent units and the one or more PCO units with respect to each other are also within the scope of the invention.

### EXAMPLES

### Example 1

A PCO unit comprising two parallel PC panels was constructed. Each PC panel was 20×24 inches in cross section, and the two PC panels were spaced five inches apart. The panels were coated with photocatalyst as described in commonly assigned U.S. patent application Ser. No. 10/345,022, filed Jan. 14, 2003, the disclosure of which is incorporated by reference herein in its entirety. Two 36 W UV lamps were positioned midway between the two PC panels. An air stream containing acetaldehyde (inlet concentrations in the range of 0.147 to 0.331 ppmw) was passed through the PCO unit. The air temperature of the air stream was in the range of 79-85° F., and the relative humidity was in the range of 35-48%.

Sampling ports were arranged so as to allow sampling of the air stream at both the inlet and the outlet of the PCO unit. The air so sampled was pumped to the injection port of a gas chromatograph for analysis of acetaldehyde concentration. Samples were taken from the outlet both when the ultraviolet lamps were turned on and when they were turned off. Table 2 shows average values of several measurements for each condition. The % removal of acetaldehyde via photocatalytic oxidation was calculated by comparing the “lights OFF” and “lights ON” conditions. The results are shown in Table 2. The percentage removal of acetaldehyde from the air stream by the PCO unit was higher when the flow rate of the air stream was lower.

TABLE 2
Removal of Acetaldehyde from an Air Stream via a PCO Unit having
Two PC panels

| | | | | Outlet | | | |
|---|---|---|---|---|---|---|---|
| | | | | Conc. | Outlet | | |
| Adjusted | | Relative | Inlet | lights | Conc. | | |
| Flow | Temp. | Humidity | Conc. | OFF | lights ON | | |
| Rate | (° F.) | (%) | (ppmw) | (ppmw) | (ppmw) | % Removal | +/− |
| 250 | 85 | 35 | 0.316 | 0.275 | 0.210 | 23.79% | 4.51% |
| 500 | 79 | 44 | 0.331 | 0.292 | 0.262 | 10.41% | 2.44% |
| 500 | 83 | 48 | 0.275 | 0.256 | 0.226 | 11.82% | 3.39% |
| 1000 | 85 | 35 | 0.147 | 0.143 | 0.130 | 9.62% | 2.62% |

### Example 2

A PCO unit comprising five parallel PC panels was constructed. Each panel was 20×24 inches in cross section, and the PC panels were spaced three inches apart. Two 36 W UV lamps were mounted between each successive pair of PC panels for a total of eight UV lamps. An air stream contaminated with low concentrations of acetaldehyde was passed through the PCO unit. The air temperature was 85° F., and the relative humidity was 21%. Air was sampled and analyzed by gas chromatography, essentially as described in Example 1. Table 3 reports average values of several measurements for each condition. The % removal of acetaldehyde from the air stream was calculated by comparing the “lights OFF” and “lights ON” conditions.

TABLE 3
Removal of Acetaldehyde from an Air Stream via a PCO Unit having
Five PC panels

| Outlet conc. | Outlet conc. | | |
|---|---|---|---|
| lights OFF, | lights ON, | | |
| (ppmw) | (ppmw) | % Removal | +/− |
| 0.732 | 0.645 | 11.86% | 1.82% |
| 0.545 | 0.459 | 15.75% | 3.25% |
| 0.365 | 0.321 | 12.13% | 1.88% |
| 0.291 | 0.222 | 23.70% | 11.67% |
| 0.205 | 0.121 | 40.97% | 5.74% |
| 0.196 | 0.165 | 15.59% | 6.18% |
| 0.195 | 0.152 | 21.87% | 11.14% |
| 0.194 | 0.130 | 33.09% | 15.94% |
| 0.162 | 0.121 | 25.18% | 9.48% |
| 0.151 | 0.108 | 60.06% | 20.21% |

Higher concentrations of acetaldehyde in the air stream generally correspond to lower percent removal of acetaldehyde. Comparing the results in Table 3 with those in Table 2 of Example 1, the PCO unit having five PC panels provides higher percentage removal of acetaldehyde as compared with the PCO unit having two PC panels.

### Example 3

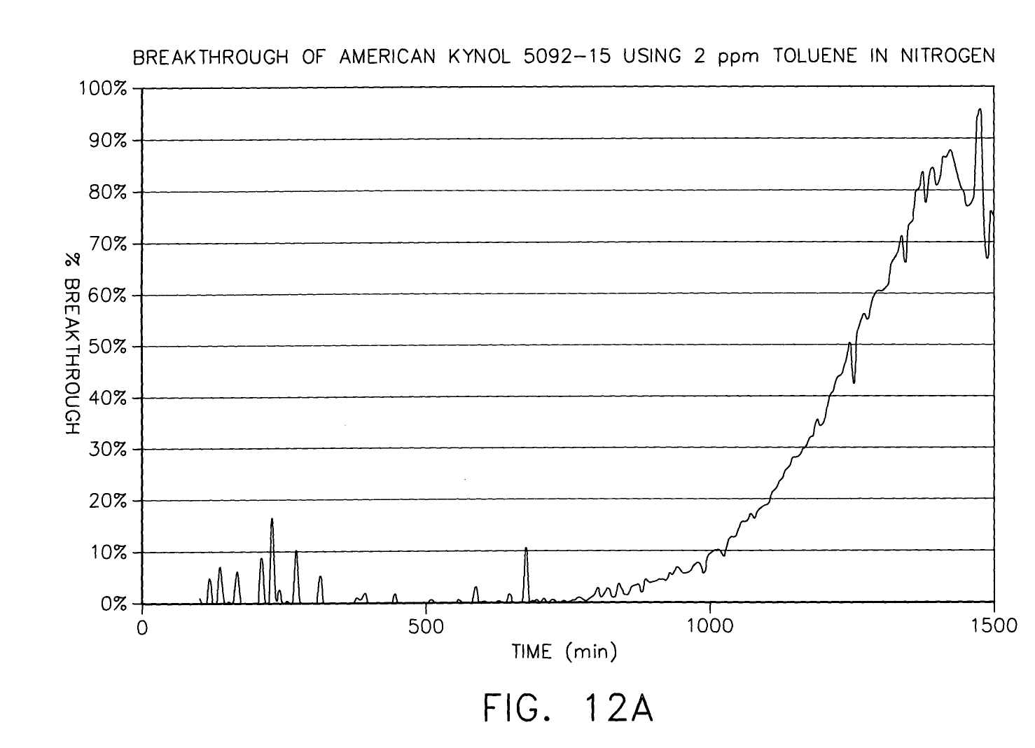

FIG. 10 shows the results of a number of experiments performed using the PCO units described in Examples 1 and 2. The flow rate of the air stream for these experiments was 500 cfm. For each experiment, the % removal of acetaldehyde from the air stream was normalized by the number of PC panels to express the data as fractional removal/panel. The equation used for this normalization was:

$$Fractional removal \text{/} panel = 1 - {ⅇ}^{( \frac{ln ( 1 - R )}{P} )} ,$$

It is apparent from FIG. 10 that fractional removal per panel tends to be higher at lower concentrations of acetaldehyde, and lower at higher concentrations. A PCO unit of the type described in Examples 1 and 2 will therefore function well with a steady low concentration of pollutant in the feed air stream. Such a feed may be experienced, for example, when an upstream adsorbent unit initially adsorbs any pulse(s) of pollutant, and subsequently desorbs the pollutant to the PCO unit at a manageable rate, generally as described hereinabove.

### Example 4

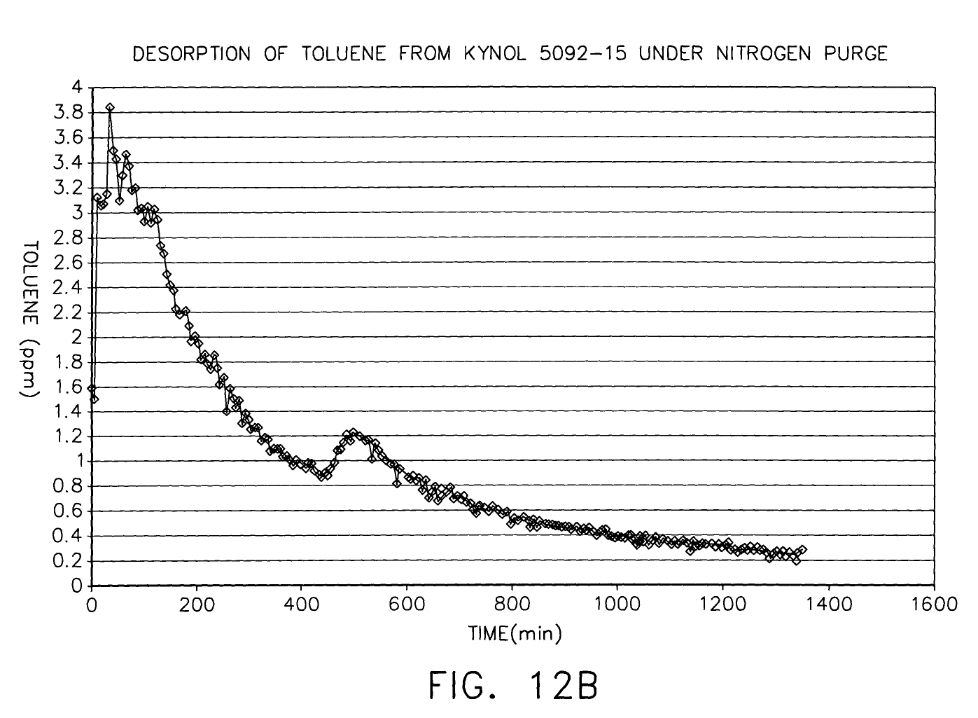

0.8826 g of activated carbon fiber adsorbent (ACF1300 obtained from Carbon Resources, Huntington Beach, Calif.), in the form of five 3.0 cm diameter discs, was degassed overnight under 28″ vacuum at 230° C. prior to placement in a 3.0 cm diameter gas phase reactor. A sample containing 529 ppm toluene in nitrogen feed gas (Scott Specialty Gas, Plumsteadville, Pa.) was blended with compressed nitrogen to create a 2 ppm toluene gas feed. Under bypass conditions, the feed was analyzed with a zNnose™ Model 4100 Gas Chromatograph (Electronic Sensor Technology, Newbury Park, Calif.) to calibrate the instrument to 2 ppm. Initially, samples were taken every six minutes for analysis by the zNose™ sensor to evaluate the adsorption capacity of the carbon adsorbent being tested. As shown in FIG. 11, after 500 minutes, a sharp breakthrough curve was apparent, and the test was concluded after it was determined that the majority of the toluene in the feed gas was not being adsorbed by the carbon adsorbent. Calculation of adsorption rates indicated adsorption of 0.00035 g toluene/min/g carbon on the fresh carbon surface.

### Example 5

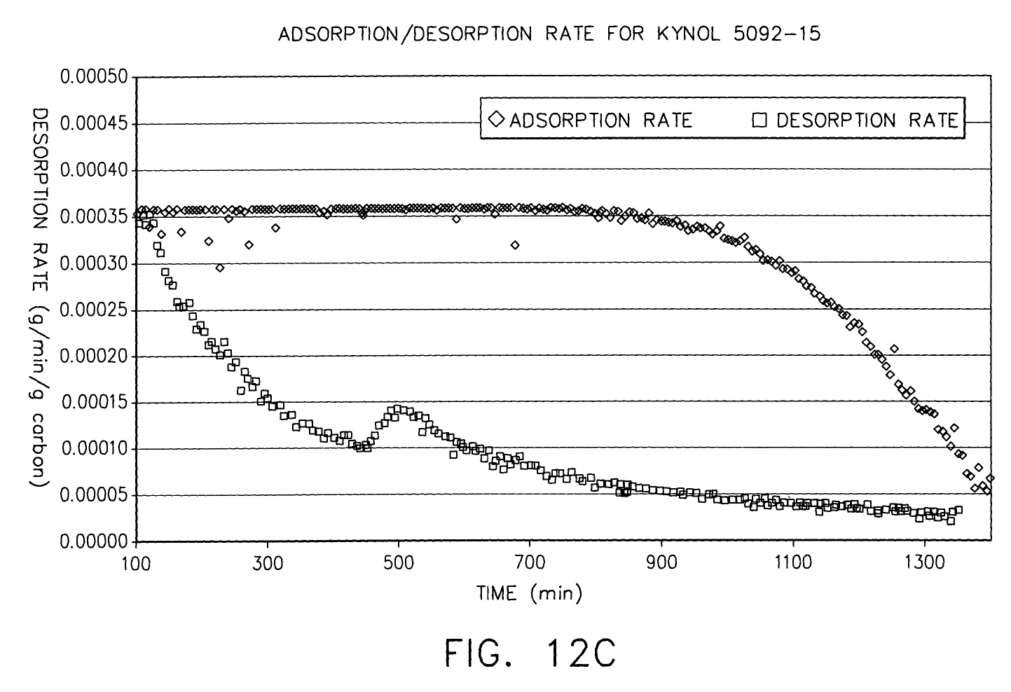

0.8473 g of carbon adsorbent (Kynol™ activated carbon fabric ACC-5092-15, obtained from American Kynol, Pleasantville, N.Y.), in the form of five 3.0 cm diameter discs, was degassed overnight under 28″ vacuum at 230° C. prior to placement in a 3.0 cm diameter gas phase reactor. A sample of 529 ppm toluene in nitrogen feed gas (Scott Specialty Gas, Plumsteadville, Pa.) was blended with compressed nitrogen to create a 2 ppm toluene gas feed. Under bypass conditions, the feed was analyzed with a zNnose™ Model 4100 Gas Chromatograph (Electronic Sensor Technology, Newbury Park, Calif.) to calibrate the instrument to 2 ppm. Initially, samples were taken every six minutes for analysis by the zNose™ sensor to evaluate the adsorption capacity of the carbon adsorbent being tested. As shown in FIG. 12A, after about 800 minutes a breakthrough curve was apparent, and the test was concluded when it was determined that the majority of the toluene in the feed gas was not being adsorbed by the carbon adsorbent.

Following the adsorption of toluene by the adsorbent as described above, a desorption curve was obtained by passing a stream of toluene-free nitrogen gas through the adsorbent, and analyzing the effluent with the zNose™ analyzer. The kinetics of toluene desorption are shown in FIG. 12B.

Using the data obtained as described above, the rates of adsorption and desorption of toluene were obtained and defined for the carbon adsorbent Kynol 5092-15 over the course of the adsorption/desorption cycle, as shown in FIG. 12C. The data demonstrate the ability of certain adsorbent materials to adsorb a pollutant at a first concentration of the pollutant and to subsequently desorb the adsorbed pollutant at a second lower concentration of the pollutant, generally as described hereinabove.

Although certain embodiments of the invention have been described primarily with respect to supplying cleansed air to an interior space of an aircraft, the invention may be equally applicable to other vehicles, buildings, etc.

It should be understood, of course, that the foregoing relates to embodiments of the invention and that modifications may be made without departing from the spirit and scope of the invention as set forth in the following claims.

1. An air quality system for removing a pollutant from an air stream, and for providing cleansed air to an interior air space, said air quality system comprising:
at least one air cleaner unit in communication with said interior air space,
wherein said at least one air cleaner unit provides only a single flow path for said air stream,
wherein said at least one air cleaner unit comprises a first photocatalytic oxidation unit and a first adsorbent unit, and
wherein said first photocatalytic oxidation unit is located upstream or downstream from said first adsorbent unit.

2. The air quality system of claim 1, wherein said first photocatalytic oxidation unit is physically separated from said first adsorbent unit.

3. The air quality system of claim 1, wherein said first photocatalytic oxidation unit is located downstream from said first adsorbent unit.

4. The air quality system of claim 3, wherein:
said first adsorbent unit is adapted to reversibly adsorb said pollutant from said air stream at a first concentration of said pollutant, and
said first adsorbent unit is further adapted to desorb said pollutant into said air stream at a second concentration of said pollutant.

5. The air quality system of claim 1, wherein:
said at least one air cleaner unit further comprises a second adsorbent unit,
said first photocatalytic oxidation unit is located downstream from said first adsorbent unit, and
said second adsorbent unit is located downstream from said first photocatalytic oxidation unit.

6. The air quality system of claim 5, wherein:
said first adsorbent unit is adapted to reversibly adsorb said pollutant from said air stream at a first concentration of said pollutant,
said first adsorbent unit is further adapted to desorb said pollutant into said air stream at a second concentration of said pollutant, and
said second adsorbent unit is adapted to irreversibly adsorb said pollutant from said air stream.

7. The air quality system of claim 6, wherein:
said first adsorbent unit includes a first adsorbent material having a first isotherm curve for said pollutant,
said second adsorbent unit includes a second adsorbent material having a second isotherm curve for said pollutant, and
said second isotherm curve is steeper than said first isotherm curve.

8. The air quality system of claim 5, wherein:
said at least one air cleaner unit further comprises a second photocatalytic oxidation unit, and
said second photocatalytic oxidation unit is located downstream from said second adsorbent unit.

9. The air quality system of claim 8, wherein said first photocatalytic oxidation unit comprises at least one photocatalytic panel, wherein said photocatalytic panel comprises a photocatalytic support, and wherein said photocatalytic panel comprises expanded aluminum.

10. The air quality system of claim 1, wherein:
said first adsorbent unit includes a first adsorbent material, said first adsorbent material having micropores in the range of from about 5 to 10 Å in diameter.

11. The air quality system of claim 10, wherein:
at least about 30% of said micropores have a diameter in the range of from about 5 to 10 Å.

12. The air quality system of claim 10, wherein:
said first adsorbent material comprises an activated carbon fabric.

13. The air quality system of claim 1, wherein said interior air space is within an aircraft.

14. The air quality system of claim 1, wherein said interior air space includes a cabin of an aircraft, and said air quality system further comprises:
at least one air intake unit; and
a mix manifold located downstream from said at least one air intake unit,
wherein said at least one air cleaner unit comprises a first battery of air cleaner units, and wherein said first battery of air cleaner units is located between said mix manifold and said cabin.

15. The air quality system of claim 1, wherein said at least one air cleaner unit is adapted for passage of a unidirectional air stream therethrough.

16. The air quality system of claim 1, wherein said at least one air cleaner unit is adapted for continuously providing a stream of cleansed air to a location downstream from said at least one air cleaner unit.

17. The air quality system of claim 1, further comprising a particulate filter upstream from said at least one photocatalytic oxidation unit and said at least one adsorbent unit.

18. The air quality system of claim 1, wherein said at least one air cleaner unit is adapted for operation at a constant temperature.

19. The air quality system of claim 1, wherein said at least one air cleaner unit is adapted for operation at ambient temperature.

20. An air quality system for an interior air space of an aircraft, said interior air space including a cabin, a lower plenum, and an upper plenum, and said air quality system comprising:
a mix manifold for distributing air to said interior air space;
a lower recirculation system in communication with said mix manifold;
an upper recirculation system in communication with said lower recirculation system;
at least one air intake unit in communication with said mix manifold; and
a plurality of air cleaner units in communication with said interior air space, each of said plurality of air cleaner units comprising at least one photocatalytic oxidation unit and at least one adsorbent unit, said at least one photocatalytic oxidation unit located upstream or downstream from said at least one adsorbent unit.

21. The air quality system of claim 20, wherein:
said plurality of air cleaner units comprise a first battery of air cleaner units coupled to said lower recirculation system, and
said first battery of air cleaner units adapted for receiving air directly from said lower plenum.

22. The air quality system of claim 20, wherein:
said interior air space further includes a flight deck, and
said plurality of air cleaner units comprise at least one air cleaner unit located between said air intake unit and said flight deck.

23. The air quality system of claim 20, wherein:
said upper recirculation system includes a plurality of cabin supply lines, and
said plurality of air cleaner units comprise a second battery of air cleaner units, and
said second battery of air cleaner units is coupled to said plurality of cabin supply lines.

24. The air quality system of claim 23, wherein said second battery of air cleaner units are adapted for receiving air directly from said upper plenum.

25. The air quality system of claim 20, wherein:
said air intake unit is located upstream from an ECS of said aircraft, and
said plurality of air cleaner units are located downstream from said ECS.

26. A vehicle, comprising:
an air quality system including at least one air cleaner unit and an interior air space,
said at least one air cleaner unit in communication with said interior air space,
said at least one air cleaner unit comprising a first photocatalytic oxidation unit, a first adsorbent unit, and a second adsorbent unit,
said first photocatalytic oxidation unit is located downstream from said first adsorbent unit, and
said second adsorbent unit located downstream from said first photocatalytic oxidation unit.

27. The vehicle of claim 26, wherein said at least one air cleaner unit further comprises a second photocatalytic oxidation unit, and said second photocatalytic oxidation unit is located downstream from said second adsorbent unit.

28. The vehicle of claim 26, wherein:
said first photocatalytic oxidation unit and said second photocatalytic oxidation unit each comprise at least one photocatalytic panel and at least one UV source, and
said at least one panel comprising a photocatalytic support and a photocatalytic agent disposed on said photocatalytic support.

29. The vehicle of claim 26, wherein:
said first adsorbent unit includes a first adsorbent material, and
said first adsorbent material comprises an activated carbon fabric having micropores in the range of from about 5 to 10 Å in diameter.

30. The vehicle of claim 26, wherein said interior air space includes an aircraft cabin.

31. The vehicle of claim 26, wherein:
said interior air space includes a flight deck,
said vehicle includes an air intake unit in communication with said flight deck, and
said at least one air cleaner unit is arranged between said air intake unit and said flight deck.

32. The vehicle of claim 26, further comprising an ECS, and wherein said at least one air cleaner unit is located downstream from said ECS.

33. An air cleaner unit for removing a pollutant from an air stream, comprising:
a housing;
a first photocatalytic oxidation unit arranged within said housing; and
a first adsorbent unit arranged parallel to said first photocatalytic oxidation unit in said housing, said housing defining only a single flow path for said air stream.

34. The air cleaner unit of claim 33, wherein:
said first photocatalytic oxidation unit is located upstream or downstream from said first adsorbent unit, and
said air cleaner unit is adapted for unidirectional passage of said air stream through said first photocatalytic oxidation unit and said first adsorbent unit.

35. The air cleaner unit of claim 33, further comprising a particulate filter located upstream from said first photocatalytic oxidation unit and said first adsorbent unit.

36. The air cleaner unit of claim 33, wherein:
said first adsorbent unit includes a first adsorbent material, said first adsorbent material having micropores therein, and
wherein at least about 30% of said micropores have a diameter in the range of from about 5 to 10 Å.

37. The air cleaner unit of claim 33, wherein:
said first adsorbent material comprises an activated carbon fabric.

38. The air cleaner unit of claim 33, further comprising a second adsorbent unit, wherein said first photocatalytic oxidation unit is located downstream from said first adsorbent unit, and said second adsorbent unit is located downstream from said first photocatalytic oxidation unit.

39. The air cleaner unit of claim 38, wherein:
said first adsorbent unit includes a first adsorbent material having a first isotherm curve for adsorption of said pollutant,
said second adsorbent unit includes a second adsorbent material having a second isotherm curve for adsorption of said pollutant, and
said second isotherm curve is steeper than said first isotherm curve.

40. The air cleaner unit of claim 33, wherein:
said first photocatalytic oxidation unit comprises at least one photocatalytic panel and at least one UV source, and
said photocatalytic panel comprises a photocatalytic support and a photocatalytic agent on said metal support.

41. The air cleaner unit of claim 33, wherein:
said first photocatalytic oxidation unit comprises a plurality of photocatalytic panels and a plurality of UV sources, and
said plurality of photocatalytic panels and said plurality of UV sources are arranged linearly and parallel to each other, with each of said plurality of UV sources alternating with each of said plurality of photocatalytic panels.

42. The air cleaner unit of claim 33, wherein said first adsorbent unit comprises an apparatus selected from the group consisting of packed bed of carbon, a carbon fabric, a solid carbon monolith, and a carbon-coated monolith.

43. An air cleaner unit for removing a pollutant from an air stream, comprising:
a first adsorbent unit;
a first photocatalytic oxidation unit located downstream from said first adsorbent unit;
a second adsorbent unit located downstream from said first photocatalytic oxidation unit; and
a housing,
said first adsorbent unit, said first photocatalytic oxidation unit, and said second adsorbent unit arranged parallel to each other within said housing,
said housing defining a single flow path for said air stream,
said first adsorbent unit, said first photocatalytic oxidation unit, and said second adsorbent unit arranged orthogonal to said air stream,
wherein said first photocatalytic oxidation unit comprises a plurality of photocatalytic panels, each of said plurality of photocatalytic panels comprises a photocatalytic support and a photocatalytic agent disposed on said photocatalytic support.

44. The air cleaner unit of claim 43, wherein:
said first adsorbent unit includes a first adsorbent material having a first isotherm curve for adsorption of said pollutant, and
said second adsorbent unit includes a second adsorbent material having a second isotherm curve for adsorption of said pollutant, and
wherein said second isotherm curve is steeper than said first isotherm curve.

45. A method for removing a pollutant from an air stream, the method comprising:
a) providing at least one air cleaner unit, said air cleaner unit comprising a first adsorbent unit, a first photocatalytic oxidation unit, and a second adsorbent unit, wherein said first photocatalytic oxidation unit is located downstream from said first adsorbent unit and said second adsorbent unit is located downstream from said first photocatalytic oxidation unit;
b) passing said air stream through said first adsorbent unit, said first adsorbent unit including a first adsorbent material having a first isotherm curve for adsorption of said pollutant;
c) thereafter, passing said air stream through said first photocatalytic oxidation unit; and
d) thereafter, passing said air stream through said second adsorbent unit, said second adsorbent unit including a second adsorbent material having a second isotherm curve for adsorption of said pollutant.

46. The method of claim 45, wherein said second isotherm curve is steeper than said first isotherm curve.

47. The method of claim 45, further comprising:
e) after said step d), passing said air stream through a second photocatalytic oxidation unit, said second photocatalytic oxidation unit being located downstream from said second adsorbent unit.

48. The method of claim 45, further comprising:
f) prior to said step b), passing said air stream through a HEPA filter, said HEPA filter being located upstream from said first adsorbent unit.

49. The method of claim 45, wherein said step a) comprises providing a plurality of air cleaner units, and wherein said air stream emanates from an ECS of an aircraft.

50. The method of claim 49, wherein at least one of said plurality of air cleaner units is located between an air intake unit and a mix manifold of the aircraft.

51. The method of claim 49, wherein said plurality of air cleaner units comprise a first battery of air cleaner units coupled to a lower recirculation system of said aircraft, and a second battery of air cleaner units coupled to an upper recirculation system of said aircraft.

52. The method of claim 45, wherein said first adsorbent material comprises an activated carbon fabric, and wherein said step b) comprises passing said airstream through said activated carbon fabric.

53. The method of claim 45, wherein said first adsorbent material includes micropores therein, said micropores having a diameter in the range of from about 5 to 10 Å.

54. The method of claim 45, wherein said first and second adsorbent units are maintained at a temperature in the range of from about 15 to 30° C.

55. The method of claim 45, wherein said pollutant comprises a VOC, a SVOC, a microorganism, or a virus.

56. A method for making an air cleaner unit for removing pollutants from an air stream, the method comprising:
a) providing a first photocatalytic oxidation unit;
b) providing a first adsorbent unit;
c) providing a housing for accommodating said first photocatalytic oxidation unit and said first adsorbent unit;
d) arranging said first adsorbent unit in said housing; and
e) arranging said first photocatalytic oxidation unit in said housing such that said first photocatalytic oxidation unit is located downstream or upstream from said first adsorbent unit, wherein said housing is adapted for providing a single flow path for passage of said air stream through said first photocatalytic oxidation unit and said first adsorbent unit.

57. The method of claim 56, wherein said first adsorbent material comprises an activated carbon fabric having micropores in the range of from about 5 to 10 Å in diameter.

58. The method of claim 56, wherein said step e) comprises arranging said first photocatalytic oxidation unit in said housing such that said first photocatalytic oxidation unit is located downstream from said first adsorbent unit.

59. The method of claim 56, further comprising:
f) providing a second adsorbent unit; and
g) arranging said second adsorbent unit in said housing such that said second adsorbent unit is located downstream from said first photocatalytic oxidation unit.

60. The method of claim 59, further comprising:
h) providing a second photocatalytic oxidation unit; and
i) arranging said second photocatalytic oxidation unit in said housing such that said second photocatalytic oxidation unit is located downstream from said second adsorbent unit.

61. The method of claim 56, further comprising:
j) arranging a particulate filter upstream from said first photocatalytic oxidation unit and from said first adsorbent unit.

62. The method of claim 56, wherein said housing comprises a duct of an air recirculation system of an aircraft.

63. The method of claim 56, wherein said housing is coupled to a cabin supply line of said aircraft.
